(19) United States
(12) Patent Application Publication
(10) Pub. No.: US 2007/0167806 A1
(43) Pub. Date: Jul. 19, 2007

(54) MULTI-MODALITY IMAGING AND TREATMENT

(75) Inventors: Bradford J. Wood, Potomac, MD (US); King Li, Bethesda, MD (US); Jeffrey H. Yanof, Solon, OH (US); Jochen Kruecker, Washington, DC (US); Christopher Bauer, Westlake, OH (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/563,713

(22) Filed: Nov. 28, 2006

Related U.S. Application Data
(60) Provisional application No. 60/740,159, filed on Nov. 28, 2005.
(60) Provisional application No. 60/740,160, filed on Nov. 28, 2005.
(60) Provisional application No. 60/744,042, filed on Mar. 31, 2006.

(51) Int. Cl. A61B 8/14

(52) U.S. Cl. 600/459

(57) ABSTRACT

A probe includes an ultrasound imaging transducer and a high intensity focused ultrasound (HIFU) transducer. The probe is operatively connected to a localizer which provides information indicative of the position and orientation of the probe in relation to a CT scanner. Information from the ultrasound imaging transducer and the CT scanner is used to assist in planning and performing a HIFU treatment.

### CROSS REFERENCE TO RELATED APPLICATIONS

This application claims the benefit of U.S. provisional application Ser. Nos. 60/740,159 filed Nov. 28, 2005, 60/740,160 filed Nov. 28, 2005 and 60/744,042 filed Mar. 31, 2006, all three of which are incorporated herein by reference.

### GOVERNMENT FUNDING

The invention described herein was developed with the support of the Department of Health and Human Services. The United States Government has certain rights in the invention.

### BACKGROUND

The present invention relates primarily to the field of medical imaging and treatment, and more particularly to techniques which facilitate the planning and application of a desired treatment under intra-procedural guidance. It finds particular application in computed tomography and ultrasound systems, although other modalities may also be used.

Multi-modality medical imaging can provide a more complete representation of a patient, area of disease, or target tissue of interest than an individual modality alone. The combination of a real time (i.e., substantially live) imaging modality (such as ultrasound imaging or fluoroscopy) with a pre-acquired (static) tomographic image data set (such as computed tomography, magnetic resonance, positron emission tomography, or single photon emission computed tomography) can be of particular interest since the real-time image stream is capable of displaying the functional and/or anatomical aspects of an interventional field at the time of the examination or treatment. The pre-acquired volumetric data set may provide different functional and/or anatomical information, or a higher resolution image, but not provide the temporal resolution needed to guide a treatment.

Moreover, two dimensional (2D) imaging modalities such as 2D ultrasound can have significant limitations for diagnosis and therapy guidance because of the limited field of view (i.e., the b-mode or planar presentation), areas of high acoustic impedance (such as bone) blocking the view, operator dependence (e.g., user-dependent choice of view direction and location), morphological changes due to breathing patterns, and the difficulty of reproducing a chosen image position at a later time. For instance, the dome of the liver may move in and out of the 2D ultrasound scan field with respiratory motion, whereas it may not with three dimensional (3D) ultrasound scan field. Also, display, imaging processing, and registration to enhance utility in 2D ultrasound imaging is limited. Consequently, the combination of 2D ultrasound with other imaging modalities is suboptimal. These and other factors likewise limit the utility of diagnostic ultrasound in treatment planning.

Turning now from imaging to treatment, high intensity focused ultrasound (HIFU) energy can be utilized for non-invasive, extracorporeal therapy in several ways. Continuous wave HIFU generates thermal lesions in the small (e.g., 1×3 millimeter) spatially confined focal zone of the HIFU probe. Larger lesions can be generated by adjusting the position and/or orientation of the HIFU probe in small, sequential increments. Tumors can be treated by creating overlapping lesions that cover the entire volume of the tumor. Pulsed HIFU can be used to accentuate drug delivery and gene transfection while minimizing adverse thermal or mechanical tissue effects, and shows great promise for new localized therapies.

However, the HIFU probe (i.e., the piezoelectric transducer) alone does not provide 3D images of the treatment zone, making accurate placement of the probe to accurately target tissue very difficult. While real time-diagnostic ultrasound, magnetic resonance and computed tomography imaging have each been used, standing alone, to plan and guide the deposition of HIFU energy, there remains substantial room for improvement.

### SUMMARY

Aspects of the present invention address these matters, and others.

According to a first aspect of the invention, an apparatus includes an ultrasound imaging system including an ultrasound transducer having a field of view. The ultrasound imaging system is adapted to generate substantially real time ultrasound data indicative of the interior of an object. The apparatus also includes a treatment apparatus connected to the ultrasound transducer for movement therewith, a second imaging system having a temporal resolution less than that of the ultrasound imaging system and adapted to generate second imaging system data indicative of an interior of the object, a localizer adapted to determine a relative position of the ultrasound transducer and the second imaging system , and a human readable display operatively connected to ultrasound imaging system and the second imaging system. The display presents a series of human readable images indicative of the ultrasound data and spatially corresponding human readable images indicative of the second imaging system data. The treatment apparatus is adapted to treat a treatment region located in the field of view.

According to another aspect of the invention, a method includes using a first imaging apparatus to obtain first volume space data indicative of an internal characteristic of an object under examination, positioning a probe including an imaging transducer and a treatment apparatus in a position with respect to the object, using information from the imaging transducer to generate a substantially real time stream of second volume space data indicative of an internal characteristic of the object, determining a spatial relationship between first and second volume space data, generating human readable images indicative of the stream of second volume space data and a spatially corresponding portion of the first volume space data, and repeating the steps of positioning the probe, using information from the imaging transducer, determining the spatial relationship, and generating human readable images a plurality of times.

According to another aspect of the invention, an apparatus includes an object support, means for generating first volume space data indicative of an object, means including a transducer for generating substantially real time second volume space data indicative of the object, means for depositing energy at a target. The means for depositing energy is operatively connected to the transducer for movement therewith, and the target is located in the field of view of the transducer. The apparatus also includes means for spatially registering the first and second volume space data, means generating human readable images indicative of the registered first and second volume space data and the target.

Those skilled in the art will appreciate still other aspects of the present invention upon reading an understanding the attached figures and description.

### FIGURES

The present invention is illustrated by way of example and not limitation in the figures of the accompanying drawings, in which like references indicate similar elements and in which:

FIG. 1 depicts a combined CT/ultrasound system.

FIG. 2A is a side view of a probe.

FIG. 2B is a top view of a probe.

FIG. 3 is a functional block diagram of a combined CT/ultrasound system.

FIG. 4 depicts information provided in a human readable display.

FIG. 5 depicts steps in a planning and performing a treatment.

### DESCRIPTION

In one implementation, a multi-modality imaging system includes a 3D ultrasound imaging system with a 3D ultrasound probe, a device to spatially locate or track the 3D probe location and orientation, a secondary imaging system, a system and procedure to co-register 3D image data generated by ultrasound and secondary imaging systems, a reconstruction and processing unit that generates human readable images (i.e., 3D to 2D projections) from the secondary imaging system that spatially correspond to the US image or 3D projection, and a display unit which combines and displays the co-registered 2D images in a fashion which maintains a real-time stream.

The system provides 3D ultrasound images co-registered with 3D CT images using a position-encoded articulated arm. The arm holding the US probe is integrated with the CT imaging system and delivers 3D spatial coordinates in CT image space. A one-time calibration system and procedure is used to convert the raw 3-D position signal from the arm into transformations that match image positions in the real-time ultrasound image volume with co-responding positions in the CT data set. Also, the CT table motion and deflection is accounted for in the transformations that localize the ultrasound probe in the 3D coordinate system of the CT and its associated data sets.

In one visualization embodiment, a reconstruction and processing unit computes two mutually orthogonal multi-planar reformatted (MPR) images from the CT data set that correspond to the real-time views provided by 3-D ultrasound imaging system. A display unit simultaneously displays the two projected CT and two corresponding ultrasound images on one screen, either side-by-side or in a fused display with a blending control, in a four view port display. The CT images have graphics that delineate the ultrasound field of view. These graphics help the user correlate the images in real-time. The reconstruction and processing unit receives ultrasound image parameters (zoom, image tilt, image rotation, etc) in order to generate CT images which match the ultrasound images as these parameters are adjusted by the sonographer.

In another implementation, a multi-modality imaging and treatment system includes a HIFU unit with a HIFU probe rigidly mounted on a diagnostic ultrasound imaging probe such that the diagnostic ultrasound system, with which the imaging probe is connected, produces images including graphics representing the focal zone of the HIFU probe. The combined HIFU and diagnostic probes are connected to a localization device to spatially locate or track the probe location and orientation. A calibration system and procedure are used to co-register the images generated by the ultrasound unit and the CT imaging system, based on the positional information provided by the localization device. A reconstruction unit extracts the sub-image from the CT system that spatially corresponds to the diagnostic US image from, and a display unit visualizes the corresponding ultrasound and CT images. A planning unit allows the selection and visualization of a treatment target in graphics a CT image. The graphics is intra-procedurally colorized to reflect the progress of the treatment.

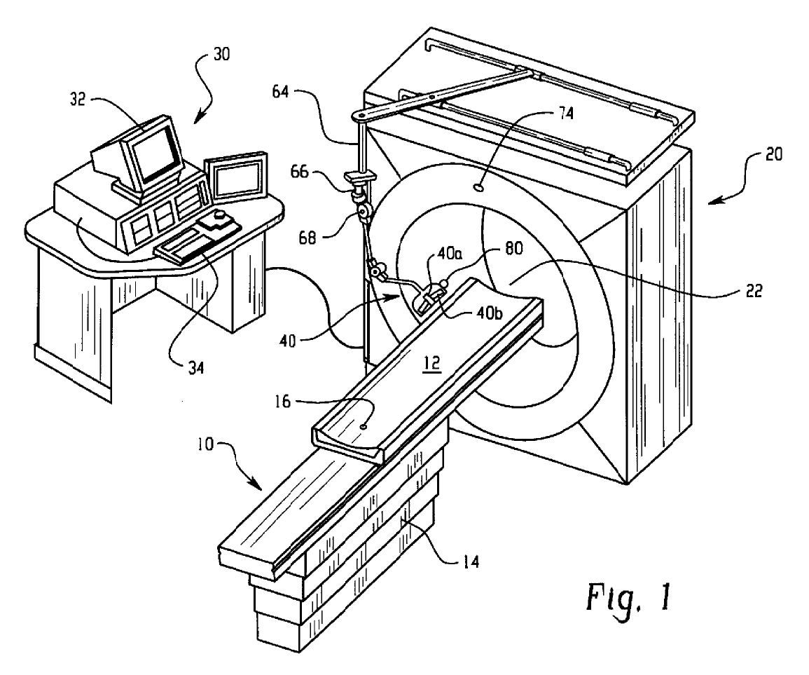

With reference to FIG. 1, an object table or support 10 includes an object supporting surface 12 that is mounted for longitudinal movement relative to a base portion 14. The base portion 14 includes a motor for raising and lowering the object support surface 12 and for moving the object support surface longitudinally. Position encoders are also provided for generating electrical signals indicative of the height and longitudinal position of the support. The support includes a calibration marker 16 disposed at a known, fixed location.

A planning, preferably volumetric diagnostic imaging apparatus 20 is disposed in axial alignment with the table 10 such that a patient or subject on the patient support surface 12 can be moved into and through an imaging region 22 of the volumetric imager. In the illustrated embodiment, the volumetric imager is a CT scanner which includes stationary and rotating gantry portions. An x-ray tube and generally arcuate radiation detector are mounted to the rotating gantry portion for rotation about the imaging region 22. The x-ray tube projects a generally cone or fan-shaped beam of radiation. X-rays which traverse the imaging region 22 are detected by the detectors, which generate a series of data lines as the rotating gantry rotates about the imaging region 22.

More specifically to the preferred embodiment, the patient support 12 moves longitudinally in coordination with the rotation of the rotating gantry so that a selected portion of the patient is scanned along a generally helical or spiral path, although generally circular or other trajectories are also contemplated. The position of the gantry is monitored by a rotational position encoder, and the longitudinal position of the patient support is monitored by a longitudinal position encoder within the support 10.

The system also includes an ultrasound imaging and HIFU systems. As will be described more fully below, an ultrasound probe 40 includes co-registered 3D US imaging 40a and HIFU 40b transducers. The position and orientation of the probe 40 are monitored by a localizer such as a mechanical arm 64 which is mounted in a known position on (or in the vicinity of the CT system 20. The arm 64 includes a plurality of arm segments 66 which are interconnected by movable pivot members 68. Encoders or position resolvers at each joint monitor the relative articulation and rotation of the arm segments. In this manner, the resolvers and encoders provide an accurate indication of the position and orientation of the probe 40 relative to the CT scanner 20.

In one implementation, the arm 64 is implemented as a passive device which is moved manually by user. Locking mechanisms such as brakes advantageously allow the user to lock the arm 64 in place using a single control or actuation when the probe 40 has been moved to a desired position. Alternately, the various joints may also be provided with suitable motors or drives connected to a suitable position control system.

A particular advantage of such an arrangement is that the arm 64 and hence the probe 40 may also be positioned under computer control.

While the above has focused on a mechanical arm 64, other localization techniques are contemplated. For example, the localization may be provided by way of optical, electro-magnetic, or sonic localization systems. Such systems generally include a plurality of transmitters and a receiver array which detects the signals from the various transmitters. The transmitters 80 (or, depending on the implementation of the localizer, the receivers) are fixedly attached to the probe 40. Their signals are used to determine the position and orientation of the probe 40.

Reconstructors associated with the CT and US imaging systems process the respective CT and US data so as to generate volumetric data indicative of the anatomy of the patient. A HIFU system likewise controls the operation of the HIFU transducer 40b.

A console 30, which typically includes one or more monitors 32 and an operator input device 34 such as a keyboard, trackball, mouse, or the like, allows a user to view volumetric images generated by, control the operation of, or otherwise interact with the imaging and HIFU portions of the system. While the console 30 has been depicted as a single console 30, it will be appreciated that separate consoles may be provided for the various imaging and treatment portions of the system.

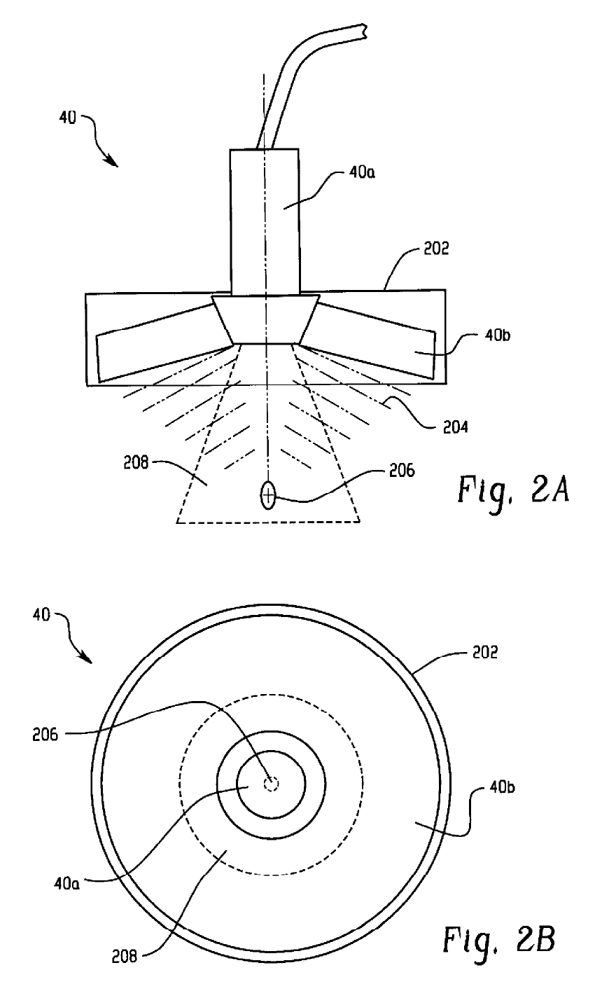

Turning now to FIGS. 2A and 2B, the ultrasound probe 40 includes a US imaging transducer 40a and a HIFU transducer 40b. As illustrated in FIGS. 2A and 2B, the transducers 40a, 40b are maintained in fixed, generally coaxial relationship by a suitable probe body 202. Also as illustrated, the HIFU transducer 40b is implemented as a generally annular transducer array which generates ultrasound energy 204 focused on a focal zone 206. The HIFU system, which is preferably connected to the console 30, allows the user to adjust the HIFU transducer 40b focal length or other parameters so as to vary the location or other characteristics of the focal zone 206.

The imaging transducer 40a, which is advantageously implemented as a conventional phased array transducer, is mounted coaxially in the center of the HIFU transducer 40b so that the focal zone 206 is located in the field of view 208 or imaging plane of the imaging transducer 40a. The ultrasound imaging system, which is also connected to the console 30, allows the user to adjust the imaging transducer 40a parameters such as zoom, image tilt, image rotation, or the like to adjust the field of view 208 or other characteristics of the ultrasound imaging system.

As will be appreciated, the volumetric data generated the CT scanner, the volumetric data generated by the US imaging system, and the HIFU transducer system are each characterized by their own spatial coordinate systems. In the system described above, however, the position and orientation of the object support 12 relative to the examination region 22 of the CT scanner 20 are known. Similarly, the mechanical arm 64 or other localizer provides information indicative of the position and orientation of the US probe 40 relative to the CT scanner 20 and hence its examination region 22. The transducers 40a, 40b likewise have a known relationship to the US probe 40. Consequently, the various coordinate systems can be correlated using known spatial coordinate correlation techniques. Provided that the patient or other object remains stationary on the support 12, the various coordinate systems likewise remain correlated to the anatomy of the patient.

As will be also appreciated, however, the accuracy of the correlation to the anatomy of the patient is influenced by factors such as gross patient motion as well as by respiratory or other periodic motion. Even in the absence of patient motion, however, the correlation accuracy is affected by factors such as the accuracy of the various position measurements, the stability and repeatability of the transducers 40a, 40b, system geometry, and similar factors. In addition, the focal zone 206 of the HIFU probe 40b is of limited spatial extent, and it is generally desirable to deposit the HIFU energy on a target region while minimizing the effects on adjacent structures. Those skilled in the art will also recognize that the CT and US scanners measure different physical parameters (radiation attenuation in the case of CT; acoustic impedance in the case of US) and thus provide different, and often complementary, information regarding the anatomy of the patient. While the CT scanner ordinarily produces images having a relatively high spatial resolution and a relatively well-defined and repeatable coordinate system, it is also characterized by a relatively poor temporal resolution. The US imaging system, on the other hand, produces images having a relatively higher temporal resolution. These characteristics can be effectively exploited in order to improve the planning and application of a HIFU energy deposition or other desired treatment.

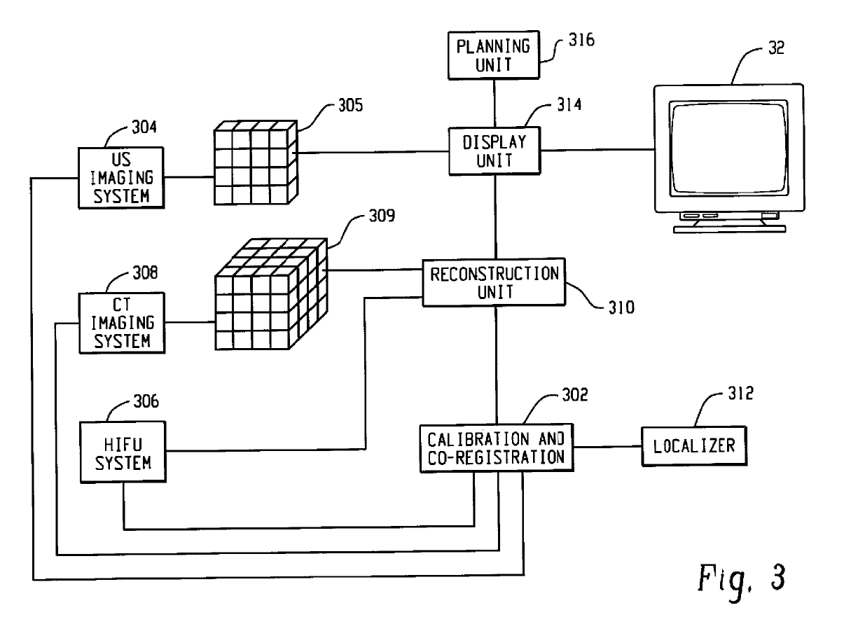

With this background, certain functional components of the system will be described in greater detail with reference to FIG. 3. The US imaging system 304 generates substantially real time volumetric data 305 having a first spatial coordinate system which is generally a function of the geometry and position of the imaging probe 40a, as well as the various probe and system settings. The CT imaging system 308 generates volumetric data 309 having a second spatial coordinate system which is generally a function of the scanner geometry and the CT imaging system 308 settings. The HIFU system 306 generates ultrasound energy focused on the focal zone 206. The HIFU system is characterized by a third spatial coordinate system which is generally a function of the geometry and position of the HIFU probe 40b and various HIFU probe and system settings.

A calibration and co-registration unit 302 uses information from the localizer 310 to co-register the US imaging system, CT imaging system, and HIFU system coordinates. In this regard, it should be noted that a one-time calibration procedure is implemented to convert the raw position signal from the localizer 312 into transformations that match or correlate the CT and US coordinate systems. This may be accomplished, for example, by imaging one or more fiducial markers 16 disposed at known locations on the patient support 12. The calibration may also be repeated at various times such as prior to or during the course of a particular imaging and/or treatment session. Support 12 motion and deflections may also be accounted for as part of the transformation process based, for example, on an a priori knowledge of the support 12 structural rigidity. The co-registration is preferably updated substantially in real time or otherwise intra-procedurally so as to reflect changes in the position of the probe 40 and/or the various system settings during the course of the procedure.

A reconstruction unit 310 extracts an image or images from the CT volumetric data 309 that spatially correspond to the then-current US image(s) 305 in the US image stream. In one implementation, the reconstruction unit 310 processes the CT data 309 to generate MPR image(s) which correspond to then-current US image(s.). A planning unit allows the user to select and visualize a treatment target on one more desired CT images. The corresponding CT image(s) may also be colorized or otherwise updated during the course of a procedure to reflect those portions of patient's anatomy which have been treated during the procedure.

The display unite 314 generates human readable image(s) indicative of the corresponding CT and US images for display on the monitor 32, for example in a side-by-side or fused display. The location of the focal zone 206 may likewise be displayed on one or both of the US and CT images. As will be appreciated, the foregoing facilitates a pre-and intra-procedural registration of the various coordinate systems and for display of data from the CT imaging, US imaging, and HIFU portions of the system.

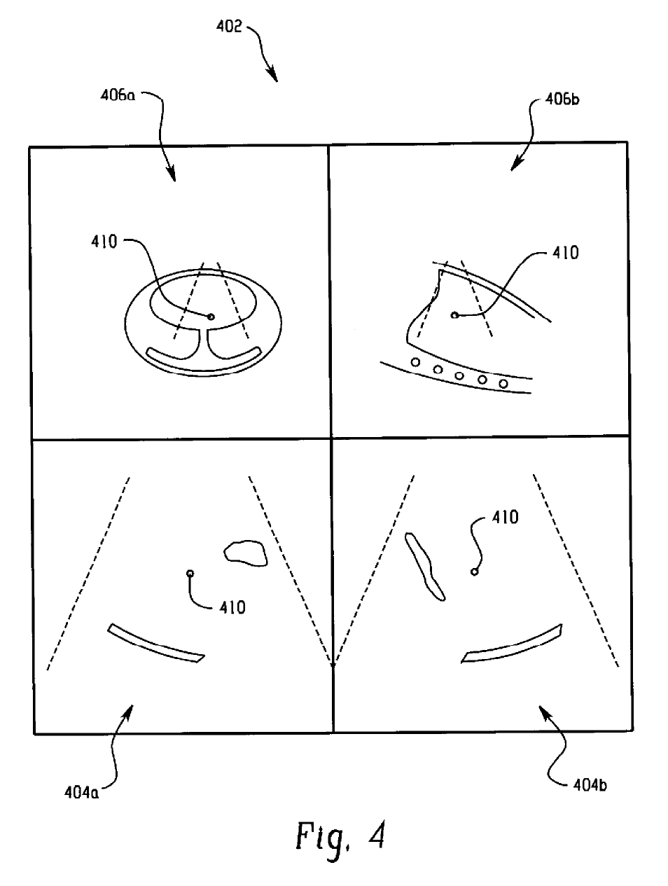

Turning now to FIG. 4, an exemplary human readable image 402 includes a four (4) port display having first 404a and second 404b US and first 406a and second 406b CT view or potts. As illustrated, the US ports 404 present orthogonal planar views of the US data 305. The first 406a and second 406b CT ports include corresponding multi-planar reformatted (MPR) images from the CT data set.

As an aid to visualization, the CT images 406 may include suitable graphics 408 which delineate the field of view of the corresponding US images 404. Similarly, suitable graphics 410 may be provided to delineate the position of the HIFU focal zone 206 and/or the target anatomy on one or both of the CT images 406 or the US images 404.

Other displays are also contemplated. For example, the corresponding images 504a, 506a and 504b, 506b may be registered and presented in fused or blended displays. A user operated blending control is advantageously provided to allow the operator to control the relative prominence of the CT and US images.

The CT images may also be presented as one more 3D rendered images which include the field of view of the US images or the focal zone 206 of the HIFU system. Again, the field of view of the US images or the focal zone 206 of the HIFU system may be delineated on the rendered images.

Once the coordinate systems have been correlated, elastic registration or other suitable techniques may be applied to account for patient motion. In one implementation, the CT data is warped to conform to the US image data at desired intervals or times during the US imaging procedure. Alternately, patient motion may be measured directly using suitable transducers. A relatively low dose multi-phasic scan of the patient can be obtained, for example at a desired number of times during the patient's respiratory cycle. For example, CT image sets may be generated at sixteen (16) or another desired number of times in the respiratory cycle. Information from the US images or the motion transducers can then be used to select the CT image set which most closely corresponds to the patient's then-current respiratory phase.

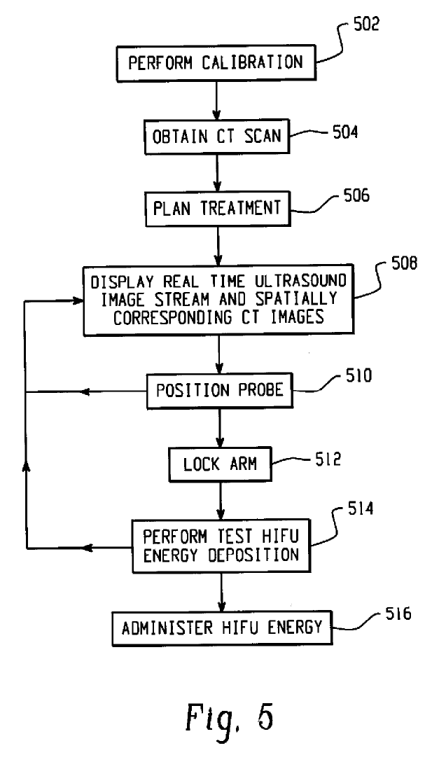

In operation, and with reference to FIG. 5, a calibration operation is performed at step 502 so as to register the CT imaging, US imaging, and HIFU coordinate systems.

A CT scan of the patient is obtained at step 504.

At step 506, the user plans the desired treatment, for example by selecting and highlighting the target area in the CT data set 309.

The real time US image stream, together with the spatially corresponding CT images and the HIFU focal zone 206, are displayed at step 508 so as to facilitate the targeting process. While it is possible to display only the CT images, co-display of the corresponding US images facilitates the detection, quantification, and correction of potential tissue) respiratory, or gross patient movement with respect to the acquired CT data.

The probe 510 is positioned at step 510. The display 508 and positioning operations 510 are repeated until the location of the HIFU focal zone 206 matches the position of the target area as depicted in the displayed images.

At step 512, the arm 64 is locked in place.

A test HIFU energy deposition may be performed at step 514. More particularly, a relatively short duration or otherwise relatively low level HIFU energy deposition is performed, and the results are displayed in the ultrasound image stream. If the observed location of the deposition does not match that of the target, the arm is unlocked and the process returns to step 508.

The desired HIFU energy is applied at step 516, for example to provide a desired thermal (ablative) treatment, for gene transfection, enhanced local drug delivery, or the like. To improve the accuracy of the HIFU energy delivery, the ultrasound imaging system may be used to provide intra-procedural feedback as to the accuracy and progress of the HIFU energy deposition. This can be accomplished, for example, by visualizing the thermal lesion, detecting physiological or other patient motion at one or more times during the energy deposition process, or by providing a respiratory or other gated HIFU energy delivery, either alone or in combination.

Other variations are possible. For example, the localizer may be implemented as an active robotic arm, and a degassed water bolus or other suitable acoustic coupling technique can be used to provide the requisite coupling between the probe 40 and the anatomy of the patient. Use of an active arm facilitates the automatic positioning of the probe, for example to match a target location identified in the CT images, repositioning the probe 40, or repeating the treatment of a desired location so as to cover a target area which is otherwise larger than the focal zone 206 of the HIFU probe 40b. Automatic correction for patient motion based on the real time ultrasound image stream is also facilitated. More particularly, suitable image processing techniques can be used to detect motion in the US image, with the information used to move the arm 64 so that the focal zone 206 remains positioned at the target.

Either 2D or 3D US imaging systems may be used. A 3D system ordinarily provides a more complete real-time visualization of the target tissue. Three dimensional, rather than 2D, motion correction is also facilitated, especially where the probe 40 is mounted to an active robotic arm. The reconstruction unit 310 can be used to provide a plurality of corresponding cross-sectional or projection images from the corresponding volumetric data 305, 309.

While the planning system 20 has been described in relation to a CT scanner, other imaging systems such as combined PET/CT, SPECT/CT, PET, or MR systems can be used. The planning system 20 may also be implemented as a real time 2D imaging modality such as fluoroscopy or CT fluoroscopy, in which case the reconstruction unit 310 extracts ultrasound images which overlap the real-time 2D image. Another real time imaging modality such as a fluoroscopy system may also be used in place of, or in conjunction with, the ultrasound imaging system.

It will also be appreciated that other probe 40 implementations are contemplated. While it is generally desirable that the imaging transducer 40a field of view 208 include the HIFU probe 40b focal zone 206, the transducers may not be located co-axially and may be disposed in other suitable relationships. The transducers may also be physically separate and provided with their own localization systems, in which case the coordinate transformations for each can be provided as described above. Moreover, the imaging 40a and HIFU 40b transducers may be implemented in a single transducer, particularly in applications such as targeted drug delivery where relatively limited HIFU energy is required.

Of course, modifications and alterations will occur to others upon reading and understanding the preceding description. It is intended that the invention be construed as including all such modifications and alterations insofar as they come within the scope of the appended claims or the equivalents thereof.

What is claimed is:

1. An apparatus including:
an ultrasound imaging system including an ultrasound transducer having a field of view and adapted to generate substantially real time ultrasound data indicative of the interior of an object;
a treatment apparatus connected to the ultrasound transducer for movement therewith, wherein the treatment apparatus is adapted to treat a treatment region located in the field of view;
a second imaging system having a temporal resolution less than that of the ultrasound imaging system and adapted to generate second imaging system data indicative of an interior of the object;
a localizer adapted to determine a relative position of the ultrasound transducer and the second imaging system;
a human readable display operatively connected to ultrasound imaging system and the second imaging system, wherein the display presents a series of human readable images indicative of the ultrasound data and spatially corresponding human readable images indicative of the second imaging system data.

2. The apparatus of claim 1 wherein the treatment apparatus includes a HIFU transducer.

3. The apparatus of claim 2 wherein the HIFU transducer and the ultrasound transducer are disposed in a probe.

4. The apparatus of claim 3 wherein the HIFU transducer and the ultrasound transducer are disposed in a coaxial relationship.

5. The apparatus of claim 2 wherein the second imaging system includes a CT system.

6. The apparatus of claim 2 wherein the HIFU transducer generates energy for deposition at the treatment region and wherein a location of the treatment region is displayed on the human readable images indicative of the second imaging system data.

7. The apparatus of claim 1 wherein the object is characterized by a periodic motion, wherein the second imaging system generates second imaging system data corresponding to each of a plurality of phases of the object motion, and wherein the display presents the series of human readable images of the ultrasound data and physically corresponding human readable images indicative of the second imaging system data.

8. The apparatus of claim 1 wherein the ultrasound imaging system, the treatment system, and the second imaging system are characterized by respective spatial coordinate systems and wherein the apparatus includes:
means for registering the coordinate systems;
means for extracting the spatially corresponding human readable images from the second imaging system data.

9. The apparatus of claim 8 wherein the localizer includes a mechanical arm operatively connected to the ultrasound transducer.

10. A method including:
using a first imaging apparatus to obtain first volume space data indicative of an internal characteristic of an object under examination;
positioning a probe including an imaging transducer and a treatment apparatus in a position with respect to the object;
using information from the imaging transducer to generate a substantially real time stream of second volume space data indicative of an internal characteristic of the object;
determining a spatial relationship between first and second volume space data;
generating human readable images indicative of the stream of second volume space data and a spatially corresponding portion of the first volume space data;
repeating the steps of positioning the probe, using information from the imaging transducer, determining the spatial relationship, and generating human readable images a plurality of times.

11. The method of claim 10 wherein the treatment apparatus is used to apply a treatment at a treatment region, and wherein the method includes indicating the position of the treatment region on the human readable images.

12. The method of claim 11 including using the first volume space data to identify a treatment target and indicating a position of the target on the human readable images.

13. The method of claim 10 wherein the treatment apparatus applies ultrasound energy to the treatment region.

14. The method of claim 13 including:
using the treatment apparatus to apply ultrasound energy to the treatment region;
using human readable images indicative of the stream of second volume space data to evaluate a result of the treatment;
adjusting a characteristic of the treatment apparatus as a function of the results of the evaluation;
using the treatment apparatus to apply additional ultrasound energy to the treatment region.

15. The method of claim 14 including indicating on the human readable images a region to which the ultrasound energy has been applied.

16. The method of claim 10 wherein the treatment apparatus causes localized heating of a treatment region.

17. The method of claim 10 including adjusting the first volume space data in response to a periodic motion of the object.

18. The method of claim 17 wherein adjusting includes warping the first volume space data.

19. The method of claim 17 wherein adjusting includes selecting first volume space data which corresponds to a phase of the periodic motion.

20. The method of claim 10 wherein the step of generating includes generating a blended image indicative of the first and second volume space data.

21. The method of claim 10 wherein the treatment apparatus includes a HIFU transducer.

22. The method of claim 21 wherein the imaging transducer includes a 3D ultrasound transducer.

23. An apparatus comprising:
an object support;
means for generating first volume space data indicative of an object;
means including a transducer for generating substantially real time second volume space data indicative of the object, and wherein the transducer includes a field of view;
means for depositing energy at a target, wherein the means for depositing energy is operatively connected to the transducer for movement therewith, and wherein the target is located in the field of view;
means for spatially registering the first and second volume space data;
means generating human readable images indicative of the registered first and second volume space data and the target.
